(19) United States
(12) Patent Application Publication
(10) Pub. No.: US 2002/0136739 A1
(43) Pub. Date: Sep. 26, 2002

(54) Subunit respiratory syncytial virus preparation

(76) Inventors: George A. Cates, Richmond Hill (CA); Sonia E. Sanhueza, Toronto (CA); Raymond P. Oomen, Aurora (CA); Michel H. Klein, Toronto (CA)

Correspondence Address: SIM & MCBURNEY, 330 UNIVERSITY AVENUE, 6TH FLOOR, TORONTO, ON, M5G 1R7, CA

(21) Appl. No.: 09/950,655

(22) Filed: Sep. 13, 2001

Related U.S. Application Data
(63) Continuation-in-part of application No. 09/214,605, filed on May 3, 1999, now Pat. No. 6,309,649.
Application No. 09/214,605 is a continuation-in-part of application No. 08/679,060, filed on Jul. 12, 1996, now Pat. No. 6,020,182.
Application No. 09/214,605 is a continuation-in-part of application No. PCT/CA97/00497, filed on Jul. 11, 1997.

(51) Int. Cl. A61K 39/12; A61K 39/155; C12N 7/00; C12N 7/01; C12P 21/08; C07K 1/00; G01N 33/561; C07K 16/00; C12Q 1/70

(52) U.S. Cl. 424/211.1; 424/204.1; 436/516; 435/5; 435/235.1; 530/388.3; 530/826

(57) ABSTRACT

The fusion (F) protein, attachment (G) protein and matrix (M) protein of respiratory syncytial virus (RSV) are isolated and purified from respiratory syncytial virus by mild detergent extraction of the proteins from concentrated virus, loading the protein onto a hydroxyapatite or other ion-exchange matrix column and eluting the protein using mild salt treatment. The F, G and M proteins, formulated as immunogenic compositions, are safe and highly immunogenic and protect relevant animal models against decreased caused by respiratory syncytial virus infection.

### REFERENCE TO RELATED APPLICATIONS

This application is a continuation-in-part of copending U.S. patent application Ser. No. 09/214,605 filed Jul. 11, 1997 which itself is a United States National Phase filing under 35 USC 371 of PCT/CA97/00497 filed Jul. 11, 1997 and a continuation-in-part of U.S. patent application Ser. No. 08/679,060 filed Jul. 12, 1996 (now U.S. Pat. No. 6,020,182).

### FIELD OF INVENTION

The present invention is related to the field of immunology and is particularly concerned with vaccine preparations against respiratory syncytial virus infection.

### BACKGROUND OF THE INVENTION

Human respiratory syncytial virus is the main cause of lower respiratory tract infections among infants and young children (refs. 1 to 3—a list of references appears at the end of the disclosure and each of the references in the list is incorporated herein by reference thereto). Globally, 65 million infections occur every year resulting in 160,000 deaths (ref. 4). In the USA alone 100,000 children may require hospitalization for pneumonia and bronchiolitis caused by RS virus it a single year (refs. 5, 6). Providing inpatient and ambulatory care for children with RS virus infections costs in excess of $340 million annually in the USA (ref. 7). Severe lower respiratory tract disease due to RS virus infection predominantly occurs in infants two to six months of age (ref. 8). Approximately 4,000 infants in the USA die each year from complications arising from severe respiratory tract disease caused by infection with RS virus and Parainfluenza type 3 virus (PIV-3). The World Health Organization (WHO) and the National Institute of Allergy and Infectious Disease (NIAID) vaccine advisory committees have ranked RS virus second only to HIV for vaccine development. Evidence is accumulating to suggest that RSV is a major cause of serious lower respiratory illness in elderly and immunocompromised adults (ref. 8A).

The structure and composition of RSV has been elucidated and is described in detail in the textbook “Fields Virology”, Fields, B. N. et al. Raven Press, N.Y. (1996), in particular, Chapter 44, pp 1313-1351 “Respiratory Syncytial Virus” by Collins, P., McIntosh, K., and Chanock, R. M. (ref. 9).

The two major protective antigens of RSV are the envelope fusion (F) and attachment (G) glycoproteins (ref. 10). The F protein is synthesized as an about 68 kDa precursor molecule (F₀) which is proteolytically cleaved into disulfide-linked F₁ (about 48 kDa) and F₂ (about 20 kDa) polypeptide fragments (ref. 11). The G protein (about 33 kDa) is heavily O-glycosylated giving rise to a glycoprotein of apparent molecular weight of about 90 kDa (ref 12). Two broad subtypes of RS virus have been defined A and B (ref. 13). The major antigenic differences between these subtypes are found in the G. glycoprotein while the F glycoprotein is more conserved (refs. 7, 14).

In addition to the antibody response generated by the F and G glycoproteins, human cytotoxic T cells produced by RSV infection have been shown to recognize the RSV F protein, matrix protein M, nucleoprotein N, small hydrophobic protein SH, and the nonstructural protein 1b (ref 15).

A safe and effective RSV vaccine is not available and is urgently needed. Approaches to the development of RS virus vaccines have included inactivation of the virus with formalin (ref. 16), isolation of cold-adapted and/or temperature-sensitive mutant viruses (ref 17) and purified F or G glycoproteins (refs. 18, 19, 20). Clinical trial results have shown that both live attenuated and formalin-inactivated vaccines failed to adequately protect vaccines against RS virus infection (refs. 21 to 23). Problems encountered with attenuated cold-adapted and/or temperature-sensitive RS virus mutants administered intranasally included clinical morbidity, genetic instability and overattenuaton (refs. 24 to 26). A live RS virus vaccine administered subcutaneously also was not efficacious (ref. 27). Inactivated RS viral vaccines have typically been prepared using formaldehyde as the inactivating agent. Murphy et al. (ref. 28) have reported data on the immune response in infants and children immunized with formalin-inactivated RS virus. Infants (2 to 6 months of age) developed a high titre of antibodies to the F glycoprotein but had a poor response to the G protein. Older individuals (7 to 40 months of age) developed titres of F and G antibodies comparable to those in children who were infected with RS virus. However, both infants and children developed a lower level of neutralizing antibodies than did individuals of comparable age with natural RS virus infections. The unbalanced immune response, with high titres of antibodies to the main immunogenic RS virus proteins F (fusion) and G (attachment) proteins but a low neutralizing antibody titre, may be in part due to alterations of important epitopes in the F and G glycoproteins by the formalin treatment. Furthermore, some infants who received the formalin-inactivated RS virus vaccine developed a more serious lower respiratory tact disease following subsequent exposure to natural RS virus than did non-immunized individuals (refs. 22, 23). The formalin-inactivated RS virus vaccines, therefore, have been deemed unacceptable for human use.

Evidence of an aberrant immune response also was seen in cotton rats immunized with formalin-inactivated RS virus (ref 29). Furthermore, evaluation of RS virus formalin-inactivated vaccine in cotton rats also showed that upon live virus challenge, immunized animals developed enhanced pulmonary histopathology (ref. 30).

The mechanism of disease potentiation caused by formalin-inactivated RS virus vaccine preparations remains to be defined but is a major obstacle in the development of an effective RS virus vaccine. The potentiation may be partly due to the action of formalin on the F and G glycoproteins. Additionally, a non-RS virus specific mechanism of disease potentiation has been suggested, in which an immunological response to contaminating cellular or serum components present in the vaccine preparation could contribute, in part, to the exacerbated disease (ref. 31). Indeed, mice and cotton rats vaccinated with a lysate of HBp-2 cells and challenged with RS virus grown on HEp-2 cells developed a heightened pulmonary inflammatory response.

Furthermore, RS virus glycoproteins purified by immunoaffinity chromatography using elution at acid pH were immunogenic and protective but also induced immunopotentiation in cotton rats (refs. 29, 32).

There clearly remains a need for immunogenic preparations, including vaccines, which are not only effective in conferring protection against disease caused by RSV but also do not produce unwanted side-effects, such as immunopotentiation. There is also a need for antigens for diagnosing RSV infection and immunogens for the generation of antibodies (including monoclonal antibodies) that specifically recognize RSV proteins for use, for example, in diagnosis of disease caused by RS virus.

### SUMMARY OF THE INVENTION

The present invention provides the production of respiratory syncytial virus (RSV) on a vaccine quality cell line, for example, VERO, MRC5 or WI38 cells, purification of the virus from fermentor harvests, extraction of the F, G and M proteins from the purified virus and copurification of the F, G and M proteins without involving immunoaffinity or lentil lectin or concanavalin A affinity steps. In particular, the lectin affinity procedure, described, for example, in WO 91/00104 (U.S. Pat. No. 07/773,949 filed Jun. 28, 1990) assigned to the assignee hereof and the disclosure of which is incorporated herein by reference), could lead to leaching of the ligand into the product.

In addition, there is provided herein, for the first time, a procedure for the coisolation and copurification of the F, G and M proteins of RSV and also immunogenic compositions comprising copurified mixtures of the RSV proteins.

The coisolated and copurified F, G and M RSV proteins are non-pyrogenic, non-immunopotentiating, and substantially free of serum and cellular contaminants. The isolated and purified proteins are immunogenic, free of any infectious RSV and other adventitious agents.

Accordingly, in one aspect of the present invention, there is provided a mixture of purified fusion (F) protein, attachment (G) protein and matrix (M) protein of respiratory syncytial virus (RSV).

The fusion (F) protein may comprise multimeric fusion (F) proteins, which may include, when analyzed under non-reducing conditions, heterodimers of molecular weight approximately 70 kDa and dimeric and trimeric forms.

The attachment (G) protein may comprise, when analyzed under non-reducing conditions, oligomeric G protein, G protein of molecular weight approximately 95 kDa and G protein of molecular weight approximately 55 kDa.

The matrix (M) protein may comprise, when analyzed under non-reducing conditions, protein of molecular weight approximately 28 to 34 kDa.

The protein mixture provided herein, when analyzed by reduced SDS-PAGE analysis, may comprise the fusion (F) protein comprising F₁ of molecular weight approximately 48 kDa and F₂ of about 23 kDa, the attachment (G) protein comprising a G protein of molecular weight approximately 95 kDa and a G protein of molecular weight approximately 55 kDa, and the matrix (M) protein comprising an M protein of approximately 31 kDa.

The mixture provided in accordance with this aspect of the invention may comprise, more preferably consists essentially of the F, G and M proteins in the relative proportions of:

F about 35 to about 70 wt %

G about 2 to about 30 wt %

M about 10 to about 50 wt %

When analyzed by SDS-PAGE under reducing conditions and densitometric scanning following silver staining, the ratio of F₁ of molecular weight approximately 48 Da to F₂ of molecular weight approximately 23 kDa in this mixture may be approximately between 1:1 and 2:1. The mixture of F, G and M proteins may have a purity of at least about 75%, preferably at least about 85%.

The mixture provided herein in accordance withis aspect of the invention, having regard to the method of isolation employed herein as described below, is devoid of monoclonal antibodies and devoid of lentil lectin and concanavalin A.

The RSV proteins provided in the mixture of proteins provided herein generally are substantially non-denatured by the mild conditions of preparation and may comprise RSV proteins from one or both of subtypes RSV A and RSV B.

In accordance with a predefined embodiment of the invention, there is provided a coisolated and copurified mixture of non-denatured proteins of respiratory syncytial virus (RSV), consisting essentially of the fusion (F) protein, attachment (G) protein and matrix (M) protein of RSV, wherein the mixture is free from lentil-lectins including concanavalin A and from monoclonal antibodies.

In accordance with another aspect of the, present invention, there is provided an immunogenic, preparation comprising an immunoeffective amount of the mixtures provided herein.

The immunogenic compositions provided herein may be formulated as a vaccine containing the F, G and M proteins for in vivo administration to a host, which may be a primate, specifically a human host, to confer protection against disease caused by RSV.

The immunogenic compositions of the invention may be formulated as microparticles, capsules, ISCOMs or liposomes. The immunogenic compositions may further comprise at least one other immunogenic or immunostimulating material, which may be at least one adjuvant or at least one immunomodulator, such as cytolcines, including IL2.

The at least one adjuvant may be selected from the group consisting of aluminum phosphate, aluminum hydroxide, QS21, Quil A or derivatives or components thereof, calcium phosphate, calcium hydroxide, zinc hydroxide, a glycolipid analog, an octodecyl ester of an amino acid, a muramyl dipeptide, polyphosphazene, a lipoprotein, ISCOM matrix, DC-Chol, DDA, and other adjuvants and bacterial toxins, components and derivatives thereof as, for example, described in U.S. application Ser. No. 08/258,228 filed Jun. 10, 1994, assigned to the assignee hereof and the disclosure of which is incorporated herein by reference thereto (WO 95/34323). Under particular circumstances, adjuvants that induce a Th1 response are desirable.

The immunogenic compositions provided herein may be formulated to comprise at least one additional immunogen, which conveniently may comprise a human parainfluenza virus (PIV) protein from PIV-1, PIV-2 and/or PIV-3, such as the PIV F and HN proteins. However, other immunogens, such as from Chlamydia, polio, hepatitis B, diphtheria toxoid, tetanus toxoid, influenza, haemophilus, B. pertussis, pneumococci, mycobacteria, hepatitis A and Moraxella also may be incorporated into the compositions, as polyvalent (combination) vaccines.

An additional aspect of the present invention provides a method of generating an immnune response in a host by administering thereto an immunoeffective amount of the immunogenic composition provided herein. Preferably, the immunogenic composition is formulated as a vaccine for in vivo administration to the host and the administration to the host, including humans, confers protection against disease caused by RSV. The immune response may be humoral or a cell-mediated immune-response.

The present invention provides, in an additional aspect thereof, a method of producing a vaccine for protection against disease caused by respiratory syncytial virus (RSV) infection, comprising administering the immunogenic composition provided herein to a test host to determine the amount of and frequency of administration thereof to confer protection against disease caused by a RSV; and formulating the immunogenic composition in a form suitable for administration to a treated host in accordance with the determined amount and frequency of administration. The treated host may be a human.

A further aspect of the invention provides a method of determining the presence in a sample of antibodies specifically reactive with an F, G or M protein of respiratory syncytial virus ISV), comprising the steps of:

(a) contacting the sample with the mixture as provided herein to produce complexes comprising a respiratory syncytial virus protein and any said antibodies present in the sample specifically reactive therewith; and

(b) determining production of the complexes.

In a further aspect of the invention, there is provided a method of determining the presence in a sample of a F, G or M protein of respiratory syncytial virus (RSV) comprising the steps of:

(a) immunizing a subject with the immunogenic composition as provided herein, to produce antibodies specific for the F, G. and M proteins of RSV;

(b) contacting the sample with the antibodies to produce complexes comprising any RSV protein present in the sample and the protein specific antibodies; and

(c) determining production of the complexes.

A further aspect of the invention provides a diagnostic kit for determining the presence of antibodies in a sample specifically reactive with a F, G or M protein of respiratory syncytial virus, comprising:

(a) a mixture as provided herein;

(b) means for contacting the mixture with the sample to produce complexes comprising a respiratory syncytial virus protein and any said antibodies present in the sample; and

(c) means for determining production of the complexes.

In an additional aspect of the invention, here is provided a method of producing monoclonal antibodies specific for F, G or M proteins of respiratory syncytial virus (RSV), comprising:

(a) administrating an immunogenic composition as provided herein to at least one mouse to produce at least one immunized mouse,

(b) removing B-lymphocytes from the at least one immunized mouse;

(c) fusing the B-lymphocytes from the at least one immunized mouse with myeloma cells, thereby producing hybridomas;

(d) cloning the hybridomas which produce a selected anti-RSV protein antibody;

(e) culturing the selected anti-RSV protein antibody-producing clones; and

(f) isolating anti-RSV protein antibodies from the selected cultures.

The present invention, in a further aspect, provides a method of producing a coisolated and copurified mixture of proteins of respirator syncytial virus, which comprises growing RSV on cells in a culture medium, separating the grown virus from the culture medium, solubilizing at least the F, G and M proteins from the separated virus; and coisolating and copurifing the solubilized RSV proteins.

The coisolation and copurification may be effected by loading the solubilized proteins onto an ion-exchange matrix, preferably a calcium phosphate matrix, specifically a hydroxyapatite matrix, and selectively coeluting the F, G and M proteins from the ion-exchange matrix. The grown virus may first be washed with urea to remove contaminants without substantially removing F, G and M proteins. Any residual DNA may be removed from the product by contacting the coeluted F, G and M proteins with an anion exchange matrix, such as Sartobind Q.

Advantages of the present invention include:

coisolated and copurified mixtures of F, G and M proteins of RSV;

immunogenic compositions containing such proteins;

procedures for isolating such proteins; and

diagnostic kits for identification of RSV and hosts infected thereby.

### BRIEF DESCRIPTION OF DRAWINGS

FIG. 1, containing panels a and b, shows SDS-PAGE analysis of a purified RSV A subunit preparation using acrylamide gels stained with silver, under both reduced (panel (a)) and non-reduced (panel (b)) conditions;

FIG. 2, containing panels a, b, c and d, shows Western blot analysis of a purified RSV subunit preparation under reduced conditions;

FIG. 3, containing panels a, b, c and d, shows Western blot analysis of a purified RSV subunit preparation under non-reduced conditions;

FIG. 4 shows SDS-PAGE analysis of a purified RSV B subunit preparation using acrylamide gels stained with silver under reduced conditions;

FIG. 5 shows a schematic flow sheet for the growth and purification of RSV subunits from infected cells; and

FIG. 6 shows a schematic flow sheet for the large scale growth and purification of RSV subunits from infected cells.

### GENERAL DESCRIPTION OF INVENTION

As discussed above, the present invention provides the F, G and M proteins of RSV coisolated and copurified from RS virus. The virus is grown on a vaccine quality cell line, such as VERO cells and human diploid cells, such as MRC5 and WI38, and the grown virus is harvested. The fermentation may be effected in the presence of fetal bovine serum (FBS) and trypsin.

The viral harvest is filtered and then concentrated, typically using tangential flow ultrafiltration with a membrane of desired molecular weight cut-off, and diafiltered. The virus harvest concentrate may be centrifiged and the supernatant. discarded, The pellet following centrifigation may first be washed with a buffer containing urea to remove soluble contaminants while leaving the F, G and M proteins substantially unaffected, and then recentrifuged. The pellet from the centrifugation then is detergent extracted to solubilize the F, G and M proteins from the pellet. Such detergent extraction may be effected by resuspending the pellet to the original harvest concentrate volume in an extraction buffer containing a detergent, such as a non-ionic detergent, including TRITON® X-100, a non-ionic detergent which is octadienyl phenol (ethylene glycol)₁₀. Other detergents include octylglucoside and Mega detergents.

Following centrifugation to remove non-soluble proteins, the F, G and M protein extract is purified by chromatographic procedures. The extract may first be applied to an ion exchange chromatography matrix to permit binding of the F, G and M proteins to the matrix while impurities are permitted to flow through the column. The ion-exchange chromatography matrix may be any desired chromatography material, particularly a calcium phosphate matrix, specifically hydroxyapatite, although other materials, such as DEAE and TMAE and others, may be used.

The bound F, G and M proteins then are coeluted from the column by a suitable eluant. The resulting codified F, G and M proteins may be further processed to increase the purity thereof

The purified F, G and M proteins employed herein may be in the form of homo and hetero oligomers including F:G heterodimers and including dimers, tetramers and higher species. The RSV protein preparations prepared following this procedure demonstrated no evidence of any adventitious agent, hemadsorbing agent or live virus.

Groups of cotton rats were immunized intramuscularly with the preparations provided herein in combination with alum or Iscomatrix as adjuvant. Strong anti-fusion and neutralization titres were obtained, as shown in Tables 1 and 2 below. Complete protection against virus infection was obtained in the upper and lower respiratory tracts, as shown in Tables 3 and 4 below.

In addition, groups of mice were immunized intramuscularly with the preparation provided herein in combination with alum, Iscomatrix polyphosphazene and DC-_{chol} as adjuvant. Strong neutralizing and anti-F antibody ditres were obtained, as shown in Tables 5 and 6 below. In addition, complete protection against virus infection was obtained, as shown by the absence of virus in lung homogenates (Table 7 below).

Groups of monkeys also were immunized with the preparations provided herein in combination with alum or Iscomatrix as adjuvant. Strong neutralizing titres and anti-F antibody titres were obtained, as shown in Tables 8 and 9 below.

The animal immunization data generated herein demonstrate that, by employing mild detergent extraction of the major RSV proteins from virus and mild salt elution of the proteins from the ion-exchange matrix, there are obtained copurified mixtures of the F, G and M RSV proteins which are capable of eliciting an immune response in experimental animals models that confers protection against RSV challenge.

The invention extends to the mixture of F, G and M proteins from respiratory syncytial virus for use as a pharmaceutical substance as an active ingredient in a vaccine against disease caused by infection with respiratory syncytial virus.

In a further aspect, the invention provides the use of A, G and M proteins from respiratory syncytial virus for the preparation of a vaccinal composition for immunization against disease caused by infection with respiratory syncytial virus.

It is clearly apparent to one skilled in the art, that the various embodiments of the present invention have many applications in the fields of vaccination, diagnosis and treatment of respiratory syncytial virus infections, and the generation of immunological agents. A further-non-limiting discussion of such issue is further presented below.

1. Vaccine Preparation and Use

Immunogenic compositions, suitable to be used as vaccines, may be prepared from mixtures comprising immunogenic F, G and M proteins of RSV as disclosed herein. The immunogenic composition elicits an immune response which produces antibodies, including anti-RSV antibodies including anti-F, anti-G and anti-M antibodies. Such antibodies may be viral neutralizing and/or anti-fusion antibodies.

Immunogenic compositions including vaccines may be prepared as injectables, as liquid solutions, suspensions or emulsions. The active immunogenic ingredient or ingredients may be mixed with pharmaceutically acceptable excipients which are compatible therewith Such excipients may include water, saline, dextrose, glycerol, ethanol, and combinations thereof The immunogenic compositions and vaccines may further contain auxiliary substances, such as wetting or emulsifying agents, pH buffering agents, or adjuvants to enhance the effectiveness thereof. Immunogenic compositions and vaccines may be administered parentally, by injection subcutaneous, intradermal or intramuscularly injection. Alternatively, the immunogenic compositions formed according to the present invention, may be formulated and delivered in a manner to evoke an immune response at mucosal surfaces. Thus, the immunogenic composition may be administered to mucosal surfaces by, for example, the nasal or oral (intragastric) routes. Alternatively, other modes of administration including suppositories and oral formulations may be desirable. For suppositories, binders and carriers may include, for example, polyalkalene glycols or triglycerides. Such suppositories may be formed from mixtures containing the active immunogenic ingredient(s) in the range of about 0.5 to about 10%, preferably about 1 to 2%. Oral formulations may include normally employed carriers such as, pharmaceutical grades of saccharine, cellulose and magnesium carbonate. These compositions can take the form of solutions, suspensions, tablets, pills, capsules, sustained release formulations or powders and contain about 1 to 95% of the active ingredient(s), preferably about 20 to about 75%.

The immunogenic preparations and vaccines are administered in a manner compatible with the dosage formulation, and in such amount as will be therapeutically effective, immunogenic and protective. The quantity to be administered depends on the subject to be treated, including, for example, the capacity of the individual's immune system to synthesize antibodies, and if needed, to produce a cell-mediated immune response. Precise amounts of active ingredient required to be administered depend on the judgment of the practitioner. However, suitable dosage ranges are readily determinable by one skilled in the art and may be of the order of micrograms to milligrams of the active ingredient(s) per vaccination. Suitable regimes for initial administration and boosted doses are also variable, but may include an initial administration followed by subsequent booster administrations. The dosage may also depend on the route of administration and will vary according to the size of the host.

The concentration of the active ingredient protein in an immunogenic composition according to the invention is in general about 1 to 95%. A vaccine which contains antigenic material of only one pathogen is a monovalent vaccine. Vaccines which contain antigenic material of several pathogens are combined vaccines and also belong to the present invention. Such combined vaccines contain, for example, material from various pathogens or from various strains of the same pathogen, or from combinations of various pathogens. In the present invention, as noted above, F, G and M proteins of RSV A and RSV B are combined in a single multivalent immunogenic composition which also may contain other immunogens.

Immunogenicity can be significantly improved if the antigens are co-administered with adjuvants. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses.

Immunostimulatory agents or adjuvants have been used for many years to improve the host immune responses to, for example, vaccines. htrinsic adjuvants, such as lipopolysaccharides, normally are the components of the killed or attenuated bacteria used as vaccines. Extrinsic adjuvants are immunomodulators which are formulated to enhance the host immune responses. Thus, adjuvants have been identified that enhance the immune response to antigens delivered parentally. Some of these adjuvants are toxic, however, and can cause undesirable side-effects, making them unsuitable for use in humans and many animals. Indeed, only aluminum-hydroxide and aluminum phosphate (collectively commonly referred to as alum) are routinely used as adjuvants in human and veterinary vaccines. The efficacy of alum in increasing antibody responses to diphtheria and tetanus toxoids is well established and a HBsAg vaccine has been adjuvanted with alum. While the usefulness of alum is well established for some applications, it has limitations. For example, alum is ineffective for influenza vaccination and usually does not elicit a cell mediated immune response. The antibodies elicited by alum-adjuvanted antigens we mainly of the IgG1 isotype in the mouse, which may not be optimal for protection by some vaccinal agents.

A wide range of extrinsic adjuvants can provoke potent immune responses to antigens. These include saponins complexed to membrane protein antigens (immune stimulating complexes), pluronic polymers with mineral oil, killed mycobacteria in mineral oil, Freund's incomplete adjuvant, bacteral products, such as muramyl dipeptide (DMP) and lipopolysaccharide (LPS), as well as lipid A, and liposomes.

To efficiently induce humoral immune responses (HIR) and cell-mediated immunity (CMI), immunogens are often emulsified in adjuvants. Many adjuvants are toxic, inducing granulomas, acute and chronic inflammations (Freund's complete adjuvant, FCA), cytolysis (saponins and Pluronic polymers) and pyrogenicity, arthritis and anterior uveitis (LPS and MDP). Although FCA is an excellent adjuvant and widely used in research, it is not licensed for use in human or veterinary vaccines because of its toxicity.

2. Immunoassays

The F, G and M proteins of RSV of the present invention are useful as immunogens for the generation of antibodies thereto, as antigens in immunoassays including enzyme-linked immunosorbent assays (ELISA), RIAs and other non-enzyme linked antibody binding assays or procedures known in the art for the detection of antibodies. In ELISA assays, the selected F, G or M protein or a mixture of proteins is immobilized onto a selected surface, for example, a surface capable of binding proteins such as the wells of a polystyrene microtiter plate. After washing to remove incompletely adsorbed material, a nonspecific protein, such as a solution of bovine serum albumin (BSA) that is known to be antigenically neutral with regard to the test sample may be bound to the selected surface. This allows for blocking of nonspecific adsorption sites on the immobilizing surface and thus reduces the background caused by nonspecific binding of proteins in the antisera onto the surface.

The immobilizing surface is then contacted with a sample, such as clinical or biological materials, to be tested in a manner conducive to immune complex (antigen/antibody) formation. This may include diluting the sample with diluents, such as solutions of BSA, bovine gamma globulin (BGG) and/or phosphate buffered saline (PBS)/Tween. The sample is then allowed to incubate for from about 2 to 4 hours, at temperatures, such as of the order of about 25° to 37° C. Following incubation, the sample-contacted surface is washed to remove non-immunocomplexed material. The washing procedure may include washing with a solution, such as PBS/Tween or a borate buffer. Following formation of specific immunocomplexes between the test sample and the bound protein, and subsequent washing, the occurrence, and even amount, of immunocomplex formation may be determined by subjecting the immunocomplex to a second antibody having specificity for the first antibody. If the test sample is of human origin, the second antibody is an antibody having specificity for human immunoglobulins and in general IgG. To provide detecting means, the second antibody may have an associated activity such as an enzymatic activity that will generate, for example, a color development upon incubating with an appropriate chromogenic substrate. Quantification may then be achieved by measuring the degree of color generation using, for example, a spectrophotometer.

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

Methods of determining tissue culture infectious dose₅₀ (TCID₅₀/mL), plaque and neutralization titres, not explicitly described in this disclosure are amply reported in the scientific literature and well within the scope of those skilled in the art. Protein concentrations were determined by the bicinchoninic acid (BCA) method as described in the Pierce Manual (23220, 23225; Pierce Chemical company, U.S.A.), incorporated herein by reference.

CMRL 1969 and Iscove's Modified Dulbeeco's Medium (IMDM) culture media were used for cell culture and virus growth. The cells used in this study are vaccine quality African green monkey kidney cells (VERO lot M6) obtained from Institut Mérieux. The RS viruses used were the RS virus subtype A (Long and A2 strains) obtained from the American Type culture Collection (ATCC), a recent subtype A clinical isolate and RSV subtype 3 clinical isolate from Baylor College of Medicine.

### EXAMPLE 1

This Example illustrates the production of RSV on a mammalian cell line on microcarrier beads in a 150 L controlled fermenter,

Vaccine quality African green monkey kidney cells (VERO) at a concentration of 10⁵ cells/mL were added to 60 L of CAL 1969 medium, pH 7.2 in a 150 L bioreactor containing 360 g of Cytodex-1 microcarrier beads and stirred for 2 hours. An additional 60 L of CMRL 1969 was added to give a total volume of 120 L. Fetal bovine serum was added to achieve a final concentration of 3.5%. Glucose was added to a final concentration of 3 g/L and L-glutamine was added to a final concentration of 0.6 g/L. Dissolved oxygen (40%), pH (7.2), agitation (36 rpm), and temperate (37° C.) were controlled. Cell growth, glucose, lactate, and glutamine levels were monitored. At day 4, the culture medium was drained from the fermenter and 100 L of E199 media (no fetal bovine serum) was added and stirred for 10 minutes. The fermentor was drained and filled again with 120 L of E199.

An RSV inoculum of RSV subtype A was added at a multiplicity of infection (M.O.I.) of 0.001 and the culture was then maintained for 3 days before one-third to one-half of the medium was drained and replaced with fresh medium. On day 6 post-infection, the stirring was stopped and the beads allowed to settle. The viral culture fluid was drained and filtered through a 20 μm filter followed by a 3 μm filter prior to firer processing.

The clarified viral harvest was concentrated 75- to 150-fold using tangential flow ultrafiltration with 300 NMWL membranes and diafiltered with phosphate buffered saline containing 10% glycerol. The viral concentrate was stored frozen at −70° C. prior to further purification.

### EXAMPLE 2

This Example illustrates the process of purifying RSV subunit from a viral concentrate of RSV subtype A.

A solution of 50% polyethylene glycol-8000 was added to an aliquot of virus concentrate prepared as described in Example 1 to give a final concentration of 6%. After sting at room temperature for one hour, the mixture was centrifuged at 15,000 RPM for 30 min in a Sorvall SS-34 rotor at 4° C. The viral pellet was suspended in 1 mM sodium phosphate, pH 6.8, 2 M urea, 0.15 M NaCl, stirred for 1 hour at room temperature, and then recentrifuged at 15,000 RPM for 30 min. in a Sorvall SS-34 rotor at 4° C. The viral pellet was then suspended in 1 mM sodium phosphate, pH 6.8, 50 mM NaCl, 1% Triton X-100 and stirred for 30 minutes at room temperature. The insoluble virus core was removed by centrifugation at 15,000 RPM for 30 min. in a Sorval SS-34 rotor at 4° C. The soluble protein supernatant was applied to a column of ceramic hydroxyapatite (type II, Bio-Rad Laboratories) and the column was ten washed with five column volumes of 1 mM sodium phosphate, pH 6.8, 50 mM NaCl, 0.02% Triton X-100. The RSV subunit composition from RSV subtype A, containing the F, G and M proteins, was obtained by eluting the column with 10 column volumes of 1 mM sodium phosphate, pH 6.8, 400 mM NaCl, 0.02% Triton X-100.

### EXAMPLE 3

This Example illustrates the analysis of RSV subunit preparation obtained from RSV subtype A by SDS polyacrylamide gel electrophoresis (SDS-PAGE) and by immunoblotting.

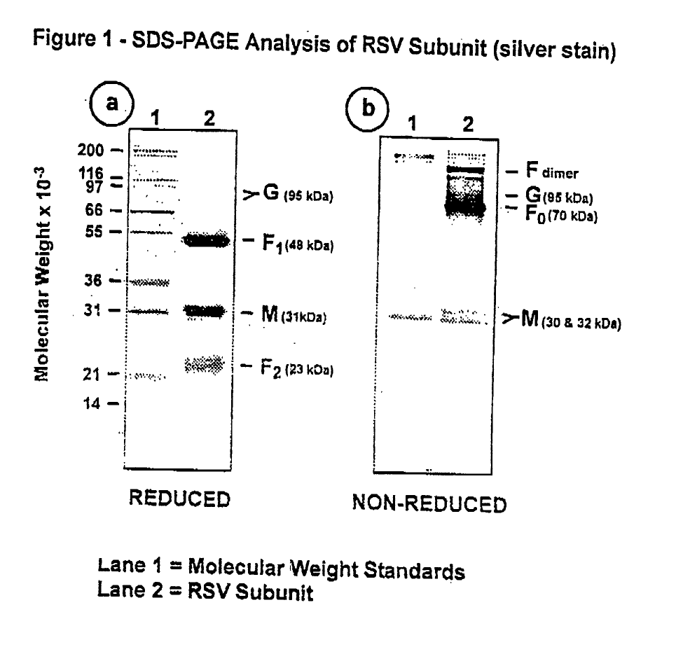

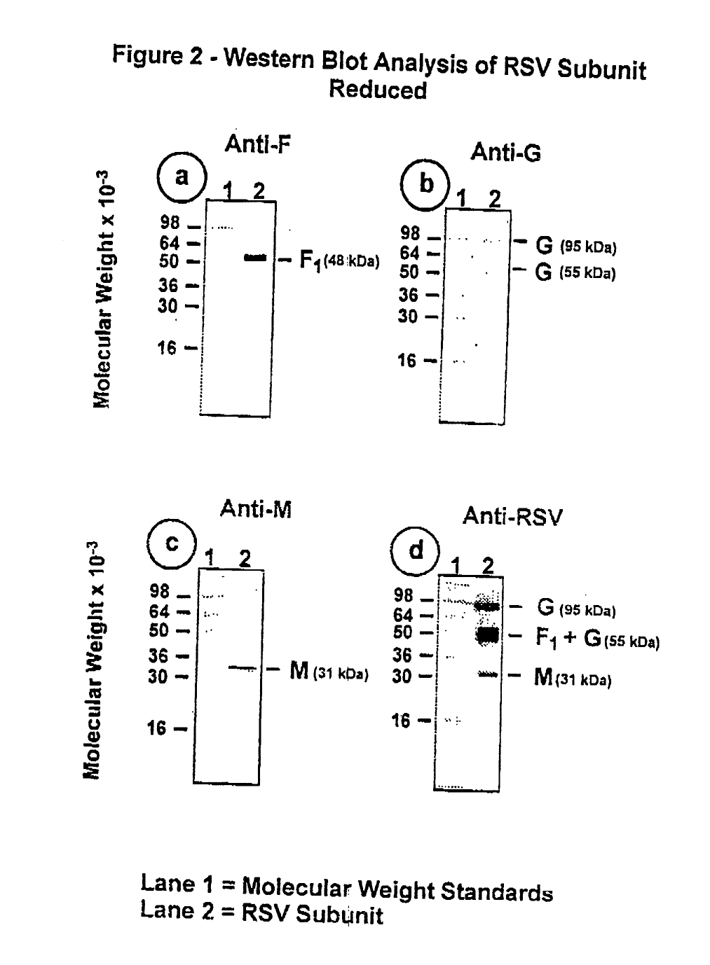

The RSV subunit composition prepared as described in Example 2 was analyzed by SDS-PAGE using 12.5% acrylamide gels. Samples were electophoresed in the presence or absence of 2-mercapteethanol (reducing agent). Gels were stained with silver stain to detect the viral proteins (FIG. 1, panels a and b). Immunoblots of replicate gels were prepared and probed with a mouse monoclonal antibody (mAb 5353C75) to F glycoprotein (FIGS. 2, panel a and 3, panel a), or a mouse monoclonal, antibody (mAb 131-2G), to G glycoprotein FIGS. 2, panel b and 3, panel b) or guinea pig anti-serum (gp178) against an RSV M peptide (peptide sequence: LKSKNMLTTVKDLTMKTLhNPTHDMIALCEFEN—SEQ ID No:1) (FIGS. 2, panel c and 3, panel c), or goat antiserum (Virostat #0605) against whole RSV (FIGS. 2, panel d and 3, panel d). Densitometric analysis of the silver-stained gel of the RSV subunit preparation electrophored under reducing conditions indicated a compositional distribution as follows:

G glycoprotein (95 kDa form)=10%

F₁ glycoprotein (48 kDa)=30%.

M protein(31 kDa)=23%

F₂ glycoprotein (23 kDa)=19%

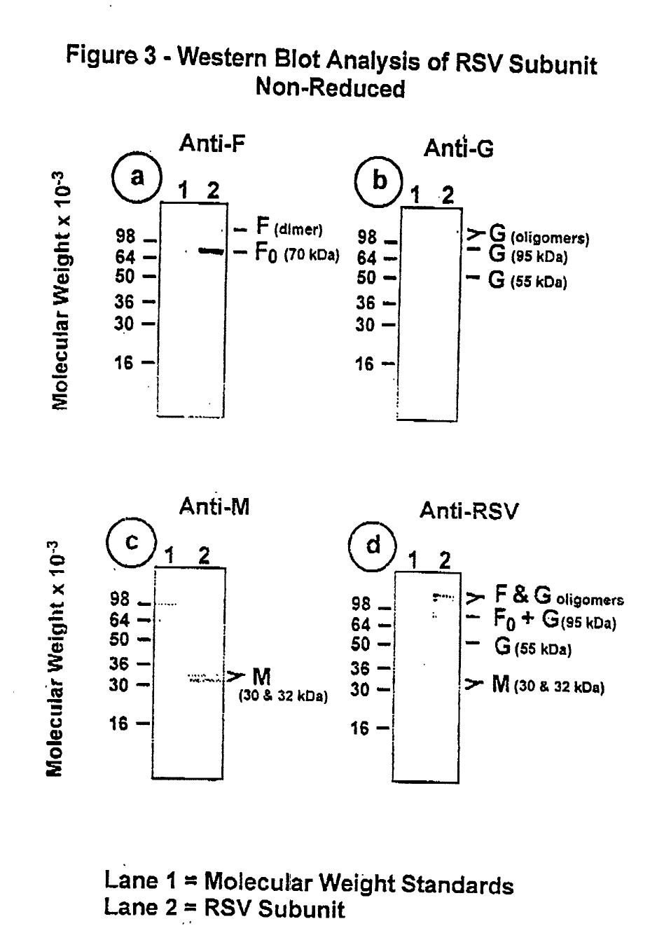

The F glycoprotein migrates under non-reducing conditions as a heterodimer of approximately 70 kDa (F₀) as well as higher oligomeric forms (dimers and trimers) (FIG. 3, panel s).

### EXAMPLE 4

This Example illustrates the immunogenicity of the RSV subunit preparation in cotton rats.

Groups of five cotton rats were immunized intramuscularly (0.1 mL) on days 0 and 28 with 1 μg or 10 μg the RSV subunit preparations produced as described in Example 2 and formulated with either 1.5 mg/dose alum or 5 μg/dose Iscomatrix™ (Iscotec, Sweden). Blood samples were obtained on day 41 and assayed for anti-fusion titres and neutralization titres. The rats were challenged intranasally on day 43 with RSV and sacrificed four days later. Lavages of the lungs and naso pharynx were collected and assayed for RV titres. Strong anti-fusion and neutralizing antibody titres were induced as shown in Tables 1 and 2 below. In addition, complete protection against virus infection was obtained with the exception of one rat, in both the upper and lower respiratory tracts (Tables 3 and 4 below),

### EXAMPLE 5

This Example illustrates the immunogenicity of the RSV subunit preparation in mice.

Groups of six BALB/c mice were immunized intramuscularly (0.1 mL) on days 0 and 28 with various doses of the RSV subunit preparation, produced as described in Example 2 and formulated with either 1.5 mg/dose alum, 10 μg/dose Iscomatrix®, 200 μg/dose polyphosphazene (PCPP) or 200 μg/dose DC-chol. The various preparations tested are set forth in Tables 5, 6 and 7 below. Blood samples were obtained on days 28 and 42 and assayed for neutralizing antibody titres and anti-F antibody titres. The mice were challenged on day 44 with RSV and sacrificed four days later. Lungs were removed and homogenized to determine virus titres. Strong neutralization titres and anti-F antibody titres were elicited as shown in Tables 5 and 6 below. In addition, complete protection against virus infection was obtained as shown by the absence of virus in lung homogenates and nasal washes (Table 7 below).

### EXAMPLE 6

This Example illustrates the immunogenicity of RSV subunit preparation in African green monkeys.

Groups of four monkeys were immunized intramuscularly (0.5 mL) on days 0 and 21 with 100 μg of the RSV subunit preparation, produced as described in Example 2 and formulated with either 1.5 mg/dose alum or 50 g/dose Iscomatrix™. Blood samples were obtained on days 21, 35 and 49 and assayed for neutralizing and anti-F antibody titres. Strong neutralizing and anti-F antibody titres were obtained as shown in Tables 8 and 9 below.

### EXAMPLE 7

This Example futher illustrates the production of RSV or a mammalian cell line or microbeads in a 150L controlled fermenter.

Vaccine quality African green monkey kidney cells (Vero cells) were added to 150L of Iscove's Modified Dulbecco's Medium (IMDM) containing 3.5% fetal bovine serum, pH 7,2, to a final concentration of 2×10⁵ cells/mL (range 1.5 to 3.5 cells/mL), in a 150 L bioreactor containing 450 g of Cytodex-1 microcarrier beads (3 g/L). Following cell inoculation, dissolved oxygen (40 percent air saturation (range 25 to 40%)), pH (7.1±0.2), agitation (36±2 rpm), and temperature (37°±0.5° C.) were controlled. Initial cell attachment to beads, cell growth (cell number determination), and growth medium levels of glucose and lactate were monitored on a daily basis. Infection of the Vero cell culture occurred three to four days following initiation of cell growth, when the concentration of cells was in the range 1.5 to 2.0×10⁶ cells/mL. Agitation was stopped and the microcarrier beads were allowed to settle for 60 minutes and the culture medium was drained from the bioreactor using a drain line placed approximately 3 cm above the settled bead volume. Seventy-five L of IMDM without fetal bovine serum (wash medium) was added and the mixture stirred at 36 rpm for 10 minutes. The agitation was stopped and the microcarrier beads allowed to settle for 30 minutes. The wash medium was removed using the drain line and then the bioreactor was filled to 75 L half volume) with IMDM without fetal bovine serum.

For infection, an RSV inoculum of RSV subtype B was added at a multiplicity of infection (M.O.I.) of 0.001 and virus adsorption to cells at half volume was carried out for 2 hours with stirring at 36 rpm. Seventy-five L of IMDM was then added to the bioreactor to a final volume of 150 L. Following infection, dissolved oxygen (40 percent air saturation (range 10-40%)), pH (7.25±0.1), agitation (36±2 rpm) and temperature (37°35 0.5° C.) were controlled. Following infection, cell growth (cell number determination) medium, glucose and lactate levels, RSV F and G antigens and RSV infectivity were monitored on a daily basis. On day 3 following infection, agitation was stopped, the microcarrier beads were allowed to settle for 60 minutes, and 75 L (50%) of the medium was removed via the drain line and replaced with fresh medium. Eight days (range seven to nine days) following infection, when complete virus-induced cytopathic effect was observed (i.e. cells were detached from the microcarrier beads, and oxygen was no longer being consumed by the culture), the agitator was stopped and the microcarrier beads were allowed to settle for 60 minutes. The virus containing culture fluid was removed from the bioreactor and transferred to a holding vessel. Seventy-five L of IMDM without fetal bovine serum was added to the bioreactor and agitated at 75 rpm for 30 minutes. The microcarrier beads were allowed to settle for 30 minutes, the rinse fluid was removed from the bioreactor and combined with the harvested material in the holding vessel.

The harvested material was concentrated approximately 20-fold by tangential flow filtration (i.e. virus-containing material was retained by the membrane) using a 500 or 1000 kilodalton (K) ultrafiltration membrane or alternatively a 0.45 μM microfiltration membrane to a final volume of 10L. The concentrated material was diafiltered with 10 volumes of phosphate-buffered saline, pH 7.2. The diafiltered viral concentrate was stored frozen at −70° C. prior to further purification.

### EXAMPLE 8

This Example illustrates the process of purifying RSV subunit from a viral concentrate of RSV subtype B.

A virus concentrate, prepared as described in Example 7, was centrifuged at 15,000 rpm for 30 man in a Sorvall SS-34 rotor at 4° C. The viral pellet was then suspended in 1 mM sodium phosphate, pH 6.8, 300 mM NaCl, 2% Triton X-100 and stirred for 36 minutes at room temperature. The insoluble virus core was removed by centrifigation at 15,000 RPM for 30 min in a Sorval SS-34 rotor at 4° C. The soluble protein supernatant was applied to a column of ceramic hydroxyapatite (type I, Bio-Rad Laboratories) and the column was then washed with ten column volumns of 1 mM sodium phosphate, pH 6.8, 10 mM NaCl, 0.02% Triton X-100. The RSV subunit composition, containing the F, G and M protein, was obtained by eluting the column with 10 column volumes of 1 mM sodium phosphate, pH 6.8, 600 mM NaCl, 0.02% Triton X-100, In some instances, the RSV subunit composition was further purified by first diluting the eluate from the first ceramic hydroxyapatite column to lower the NaCl concentration to 400 mM NaCl and then applying the diluted submit onto a column of ceramic hydroxyapatite (type II, Bio-Rad Laboratories). The flowthrough from his column is the purified RSV subunit composition from RSV subtype B.

### EXAMPLE 9

This Example illustrates the analysis of RSV subunit preparation obtained from RSV subtype B by SDS polyacryamide gel electrophoresis (SDS-PAGE).

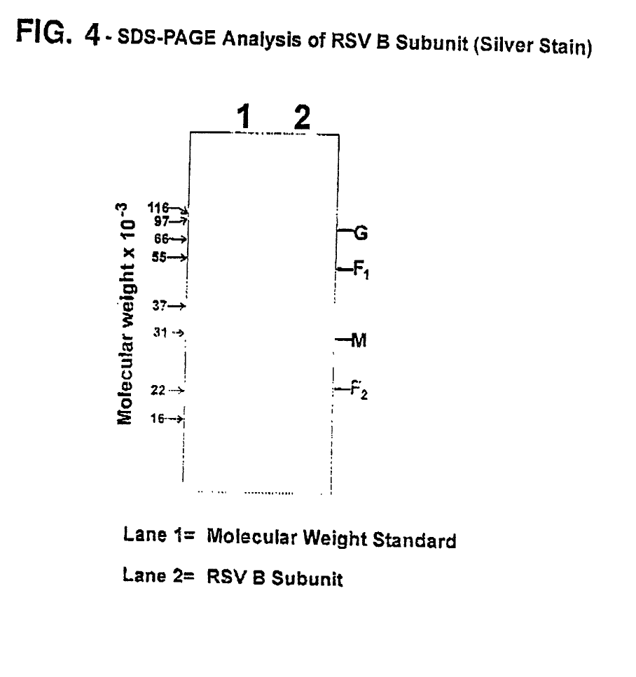

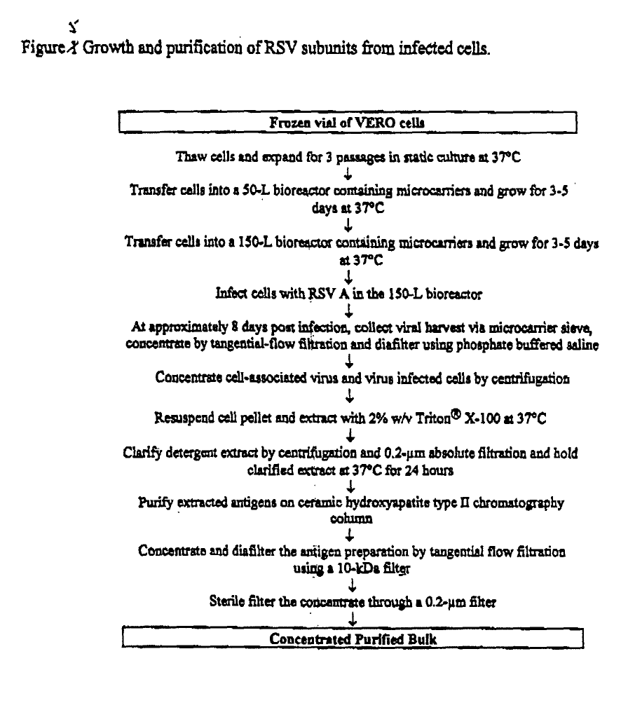

The RSV subunit composition prepared as described in Example 8 was analyzed by SDS-PAGE using a 15.0% acrylamide gel. The sample was electrophoresed in the presence of 2-mercaptoethanol (reducing agent). The gel was stained with silver stain to detect the viral proteins (FIG. 4). Densitometric analysis of the silver-stained gel of the RSV subunit preparation under reducing conditions indicated a compositional distribution of the proteins as follows:

G glycoprotein (95 kDa form)=21%

F₁ glycoprotein (48 kDa)=19%

M protein (31 kDa)=22%

F₂ glycoprotein (23 kDa)=20%

### EXAMPLE 10

This Example illustrates growing and purifying RSV sub-units from infected cells (see FIG. 5).

VERO cells (Lot LS-7) were grown for three passages in static culture at 37° C. in medium (CMRL 1969) containing 10% v/v PBS. The cells were then transferred to a 50-L bioreactor containing microcarriers and to T150 control cell flasks in medium (CMRL 1969) containing 3.5% v/v FBS and incubated for 3 to 5 days at 37° C. These cells were then transferred to a 150-L bioreactor containing microcarriers in medium containing 3.5% v/v FBS and incubated for 3 to 5 days at 37° C. After 3 to 4 days of growth at 37° C. in the 150-L bioreactor, the microcarriers are allowed to settle and the growth medium was removed. The cells were then washed once with serum-free medium and the microcarriers were allowed to settle and the medium removed. The cells were then infected with RSV A in 1500 L serum-free medium. After 3 to 4 days post-infection, the microcarriers are allowed to settle, and half of the volume of medium was replaced with serum-free medium. The cells were then incubated for a further 4 to 6 days at 37° C.

The cells were then harvested and filtered trough a 100 μm sieve and washed with 500 L of PBS. The microcarrier-free material was collected in a holding tank and concentrated by tangential flow filtration on a 500-kDa filter membrane This material was concentrated approximately 20-fold and diafiltered using Dulbecco's PBS.

The virus infected cells and cell associated virus were then collected by batch centrifugation for 30 minutes at 5,000× g. The pellet was resuspend in 10 mM sodium phosphate buffer, containing 300 mM NaCl. The resuspended pellet was then extracted with 2% w/v Triton® X-100 and stirred at 35° to 39° C. for 1 hour. The extract containing soluble F, G and M viral proteins was then clarified the extract by centrifigation for 60 min at 25,000× g. The supernatant was then diluted 3- to 5-fold with 2% w/v Triton® X-100 solution and further clarified by filtration through an absolute 0.2-μm filter.

The filtered extract was then maintained at 35 to 39° C. for 24 hours with mixing for RSV Us inactivation. To the extract. 2% w/v Triton®X-100 was added to dilute the supernatant 10-fold as compared to initial volume of supernatant. The eject containing F, G and M proteins was then loaded onto a ceramic hydroxyapatite type II chromatography column and the column equilibrated with 1 mM sodium phosphate buffer, containing 30 mM NaCl and 0.02% w/v Triton® X-100.

F, G and M proteins were then eluted with 1 mM sodium phosphate buffer, containing 550 mM NaCl and 0.02% w/V Triton® X-100 and concentrated by ultrafiltration on a 10-kDa filter membrane and diafiltered with 10 mM sodium phosphate buffer, containing 150 mM NaCl and 0.01% w/v Triton®X-100. The resulting solution containing F, G and M proteins was sterilized using a 0.2 μm absolute filter. This represents the concentrated purified bulk (FIG. 5).

The concentrated bulk had a composition distribution:

| | |
|---|---|
| F glycoprotein | 48 wt% |
| G glycoprotein | 5 wt% |
| M Protein | 42 wt% |
| Protein impurities | 5 wt% |

### EXAMPLE 11

This Example describes the formulation of vaccines and testing in humans.

RSV sub-unit preparations, produced according to Example 10, were used to formulate an alum-adjuvanted vaccine and a placebo control that contained only alum. The total protein present in a single dose of the vaccines of the antigens RSV F, G, and M was 100 μg, present in 0.5 mL of phosphate buffered saline. In the alum-adjuvanted vaccine, there was 1.5 mg of alum per 0.5 mL of vaccine,

The vaccines were assessed for stability for 42 months at 5° C., 5 months at 25° C. and 5 weeks at 37° C. to ensure physical and biological stability over time. Stability studies indicated that the P and G antigens in the alum-adjuvanted vaccines are stable at 25° C. for at least 6 weeks.

The vaccine preparations were used to immunize adults, 65 years of age or older. Blood samples were obtained on day 0 (day of immunization), day 32, day 60 and day 180, RSV serology was performed on the serum samples as follows:

RSV neutralizaon assays by a plaque reduction method (NA) against RSV A and RSV B as follows:

1. A colourmetric 96-well plaque reduction assay in tissue culture cells was performed on human sera to assess the neutralization titre. The titre is defined as the amount of human sera requited to neutralize 60% of a standard RSV A virus sample. The assay is based on Prince et al. (ref. 33).

The sera were heat-inactivated at 56° C. for 30 minutes. The samples were then diluted in 3-fold serial steps in a 96-well plates and mixed with an equal volume of RSV A (Long strain 30 to 70 pfu) in assay media containing 10% guinea pig complement.

After incubation for 1 hour at 37° C., the mixture was inoculated onto VERO cells for 1 to 2 hours. The inoculum was then removed and the VERO cells overlaid with 0.75% methyloellulose and incubated for 4 to 5 days. After the 4-day incubation, the cells were fixed with a mixture of 2% formaldehyde and 0.2% glutaraldehyde. Viral plaques were then visualized by immunostaining using a monoclonal antibody to the RSV F protein, followed by a donkey anti-mouse IgG F(ab′)2 -horseradish peroxidase conjugate. The enzyme substrates were tetramethylbenzidirine (TMB) and hydrogen peroxide. The neutralization titre is expressed as the reciprocal of the dilution which results in 60% reduction in plaque formation as determined by linear interpolation analysis (Tables 1 to 3).

2. F glycoprotein-specific antibodies were measured by enzyme linked immunoassay LISA). Enzyme linked immunosorbert assays (ELISA) are generally known in the art. Briefly, this ELISA assay is for the detection and quantitation of human IgG antibodies to the Fusion (F) protein of Respiratory Syncytial Virus A (RSVA F). The assay utilizes microtiter plates coated with purified RSV-F antigen to sequester F-specific IgG antibodies and peroxidase-coupled antibodies to human IgG as the indicator.

Microtitre plates were coated with purified RSV-F antigen for 16 to 24 hours. The coating solution was blotted, and the plates were incubated with a blocking solution and then washed. Dilutions of serum standard, control sera and test samples were added to the wells. The plates were incubated and washed. Horseradish peroxidase (HRP)-conjugated anti-human IgO was added at the working dilution. The plates were incubated and washed again. Tetramethyl benzidine (TV) was diluted to the working concentration in hydrogen peroxide (H₂O₂) was added and the plates were incubated further. The reaction was quenched with 1 M sulphuric acid (H₂SO₄) and the colour reaction measured by reading the optical density (O.D.) of each well.

In this procedure, a test sample containing IgG antibodies to RSV-F forms a 3-layer sandwich attached to the solid phase (mirotitre plate). The intensity of colour development in each well is directly proportional to the amount of anti-human IgG peroxidase attached to the solid phase and, therefore, to the anti-RSV-F IgG content of the test sample. To quantitate the amount of anti-RSV-F IgG in each test sample, eight (8) 2-fold dilutions of each sample are tested against a serially diluted standard. Two controls, a positive and a negative, are included on each plate. Antibody levels are expressed in ELISA units (E.U.), obtained by assigning 100,000 E.U. to the Serum Standard.

3. G glycoprotein-specific antibodies were measured by enzyme linked immunoassay (ELISA). Briefly, this ELISA assay is for the detection and quantitation of human IgG antibodies to the attachment glycoprotein (G) of Respiratory Syncytial Virus (RSV). The assay utilizes microtitre plates coated with purified RSV-G antigen to bind G-specific IgG antibodies and peroxidase-coupled, antibodies to human IgG as the indicator.

Microtitre plates were coated with purified RSV-G antigen for 16 to 24 hours. The coating solution was blotted, and the plates were incubated with a blocking solution and then washed. Dilutions of serum standard, control sera and test samples were added to the wells. The plates were incubated and washed. Horseradish peroxidase (HRP) conjugated anti-human IgG was added at the working dilution. The plates were incubated and washed again. Tetramethyl benzidine (TMB) diluted to the working concentration in hydrogen peroxide (H₂O₂) was added and the plates were incubated fierier. The reaction was quenched with 1M sulphuric acid (H₂SO₄) and the colour reaction measured by reading the optical density (O.D.) of each well.

In this procedure, a test sample containing IgG antibodies to RSV-G forms a 3-layer sandwich attached to the, solid phase (microtitre plate). The intensity of colour development in each well is directly proportional to the amount of anti-human IgG peroxidase attached to the solid phase and, therefore, to the anti-RSV-G IgG content of the test sample. To quantitate the amount of anti-RSV-G I&G in each test sample, eight (8) 2-fold dilutions of each sample were tested against a serially-diluted standard Two controls, a positive and a negative, were included on each plate. Antibody levels are expressed in ELISA units (E.U.), obtained by assigning 100,000 E.U. to the Serin Standard.

The immunogenicity of the vaccine preparation is shown in Table 10 as the geometric mean titer and the 95% confidence intervals for the vaccine adjuvanted with alum and the alum control.

Tables 10 and 11 show the number of vaccines in which there was a greater or equal to 2-fold increase in antibody titer (Table 11) or 4-fold increase in antibody titer (Table 12) compared to pre-immunization titers.

### EXAMPLE 12

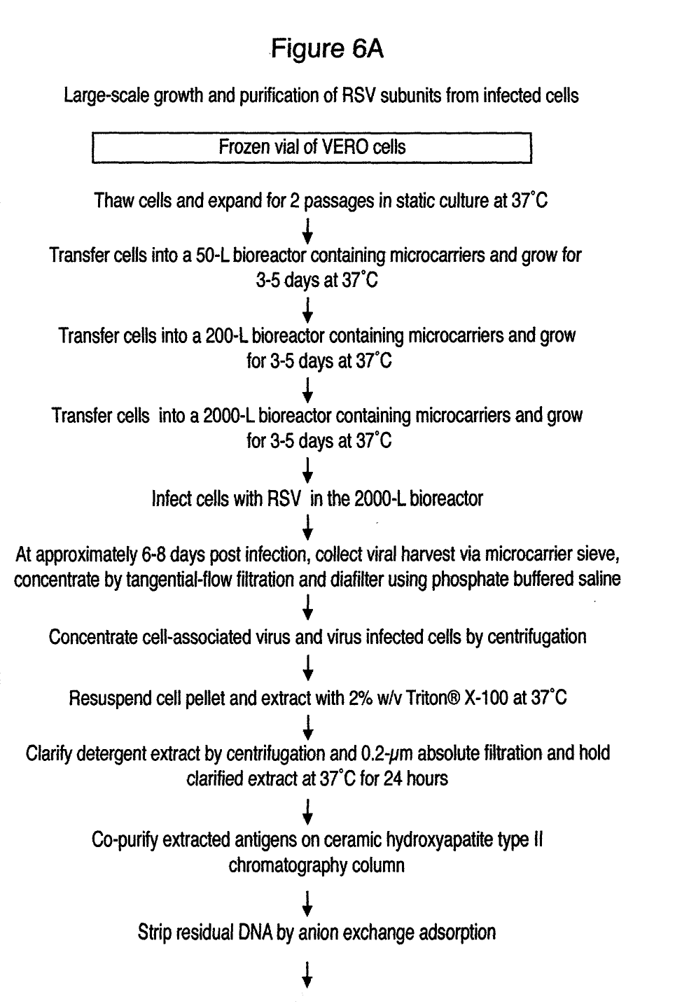

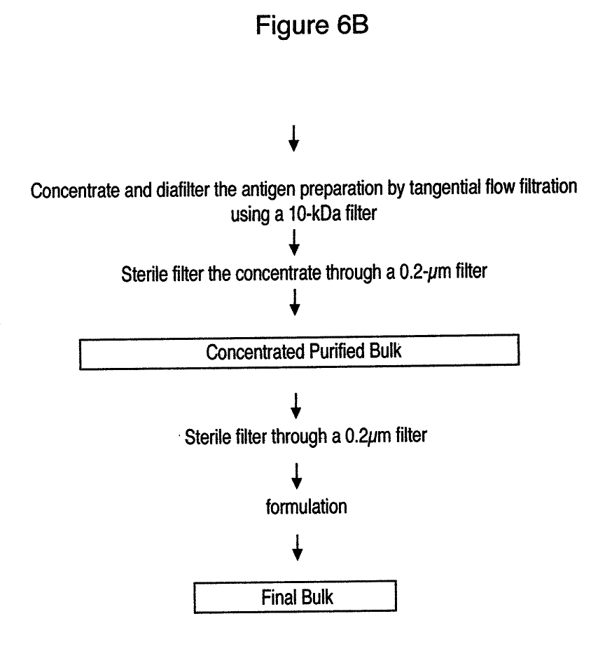

This Example illustrates large-scale growth and purification of RSV sub-units from infected cells (see FIG. 6).

VERO cells (Lot LS-7) were grown for two passages in static culture at 37° C. in medium (CMRL 1969) containing 10% v/v FBS. The cells were then transferred to a 50-L bioreactor containing microcarriers and to T150 control cell flasks in medium (CMRL 1969) containing 3,5% v/v FBS and incubated for 3 to 5 days at 37° C. These cells were then transferred to a 200-L bioreactor containing microcarriers in medium containing 3.5% V/V FBS and incubated for 3 to 5 days at 37° C. These cells were then transferred to a 2000-L bioreactor containing microcarriers and incubated for 3 to 5 days at 37° C. After 3 to 4 days of growth at 37° C. in the 200-L bioreactor, the microcarriers are allowed to settle and the growth medium was removed. The cells were then washed once with serum-free medium and the microcarriers were allowed to settle and the medium removed. The cells were then infected with RSV A. After 3 to 4 days post-infection, the microcarriers are allowed to settle.

The cells were then harvested and filtered through a 100 μm sieve and washed with PBS. The microcarrier-free material was collected in a holding tank and concentrated by tangential flow filtration on a 500-kDa filter membrane. This material was concentrated approximately 20-fold and diafiltered using Dulbecco's PBS.

The virus infected cells and cell associated virus were then collected by batch centrifugation for 30 minutes at 5,000× g. The pellet was resuspend in 10 mM sodium phosphate buffer, containing 300 mM NaCl. The resuspended pellet was then extracted with 2% w/v Triton® X-100 and stirred at 35° to 39° C. for 1 hour. The extract containing soluble F, G and M viral proteins was then clarified the extract by centrifugation for 60 min at 25,000× g. The supernatant was then diluted 3- to 5-fold with 2% w/V Triton® X-100 solution and further clarified by filtration though an absolute 0.2-μm filter.

The filtered extract was then maintained at 35 to 39° C. for 24 hours with mixing for RSV virus inactivation. To the extract, 2% w/v Triton® X-100 was added to dilute the supernatant 10-fold as compared to initial volume of supernatant. The extract containing F, G and M proteins was then loaded onto a ceramic hydroxyapatite type II chromatography column and the column equilibrated with 1 mM sodium phosphate buffer, containing 30 mM NaCl and 0.02% w/v Triton® X-100.

F, G and M proteins were then eluted with 1 mM sodium phosphate buffer, containing 550 mM NaCl, and 0.02% w/v Triton® X-100 and concentrated by ultrafiltration on a 10-kDa filter membrane and diafiltered with 10 mM sodium phosphate buffer, containing 150 mM NaCl and 0.01% w/v Triton® X-100. The resulting solution then was passed through a sartobind Q (Sartorius) chromatography column to remove residual DNA by micron-exchange adsorption. The resulting solution containing F, G and M proteins was sterilized using a 0.2 μm absolute filter. This represents the concentrated purified bulk (FIG. 6).

### SUMMARY OF DISCLOSURE

In summary of this disclosure, the present invention provides a coisolated and purified mixture of F, G and M proteins of RSV which is able to protect against RSV in relevant animal models of infection. Modifications are possible within the scope of this invention.

TABLE 1
Serum Anti-Fusion Titres in Cotton Rats

| Group | Mean titre (log₂) | Std. Dev. (log₂) |
|---|---|---|
| Alum placebo | 2.0 | 0.0 |
| Iscomatrix ™ placebo | 2.3 | 0.5 |
| RSV Subunit 1 μg with Alum | 8.0 | 1.0 |
| RSV Subunit 10 μg with Alum | 7.5 | 1.0 |
| RSV Subunit 1 μg with Iscomatrix ™ | 10.4 | 1.3 |
| RSV Subunit 10 μg with Iscomatrix ™ | 10.0 | 1.6 |

TABLE 2
Serum Neutralization Titres in Cotton Rats

| Group | Mean titre (log₂) | Std. Dev. (log₂) |
|---|---|---|
| Alum placebo | 2.0 | 0.0 |
| Iscomatrix ™ placebo | 2.0 | 0.0 |
| RSV Subunit 1μg with Alum | 9.6 | 1.3 |
| RSV Subunit 10 μg with Alum | 10.0 | 1.4 |
| RSV Subunit 1 μg with Iscomatrix ™ | 10.6 | 1.1 |
| RSV Subunit 10 μg with Iscomatrix ™ | 11.2 | 1.1 |

TABLE 3
Pulmonary Wash RSV Titres in Cotton Rats

| | Mean titre | Std. Dev. |
|---|---|---|
| Group | (log₁₀/g lung) | (log₁₀/g lung) |
| Alum placebo | 3.8 | 0.4 |
| Iscomatrix ™ placebo | 3.7 | 0.5 |
| RSV Subunit 1 μg with Alum | 0.4 | 0.8 |
| RSV Subunit 10 μg with Alum | 0.0 | 0.0 |
| RSV Subunit 1 μg with Iscomatrix ™ | 0.0 | 0.0 |
| RSV Subunit 10 μg with Iscomatrix ™ | 0.0 | 0.0 |

TABLE 4
Nasal Wash RSV Titres in Cotton Rats

| | Mean titre | Std. Dev. |
|---|---|---|
| Group | (log₁₀/g lung) | (log₁₀/g lung) |
| Alum placebo | 3.2 | 0.5 |
| Iscomatrix ™ placebo | 3.1 | 0.3 |
| RSV Subunit 1 μg with Alum | 0.0 | 0.0 |
| RSV Subunit 10 μg with Alum | 0.0 | 0.0 |
| RSV Subunit 1 μg with Iscomatrix ™ | 0.0 | 0.0 |
| RSV Subunit 10 μg with Iscomatrix ™ | 0.0 | 0.0 |

TABLE 5
Serum Neutralization Titres in Balb/c Mice

| | |
|---|---|
| 4 Week Bleed | 6 Week Bleed |

| | Mean titre | Std. Dev. | Mean titre | Std. Dev. |
|---|---|---|---|---|
| Group | (log₂) | (log₂) | (log₂) | (log₂) |
| Alum placebo | 3.0¹ | 0.0 | 3.0 | 0.0 |
| Iscomatrix ™ placebo | 3.0 | 0.0 | 3.0 | 0.0 |
| PCPP placebo (200 μg) | ND | ND | 3.0 | 0.0 |
| DC-Chol placebo (200 μg) | ND | ND | 3.0 | 0.0 |
| RSV Subunit 0.1 μg with no adjuvant | ND | ND | 3.0 | 0.0 |
| RSV Subunit 0.1 μg with Alum | ND | ND | 10.3 | 0.9 |
| RSV Subunit 1 μg with Alum | 6.5 | 0.6 | 8.7 | 1.0 |
| RSV Subunit 10 μg with Alum | 8.0 | 1.1 | 9.5 | 1.1 |
| RSV Subunit 1 μg with Iscomatrix ™ | 8.2 | 0.8 | 13.2 | 1.0 |
| RSV Subunit 10 μg with Iscomatrix ™ | 10.4 | 1.3 | 13.4 | 0.6 |
| RSV Subunit 1 μg with PCPP (200 μg) | ND | ND | 15.0 | 0.6 |
| RSV Subunit 0.5 μg with DC-Chol (200 μg) | ND | ND | 11.7 | 1.1 |

¹ minimal detectable titre in assay
ND = not determined

TABLE 6
Serum Anti-F Titres in Balb/c Mice

| | |
|---|---|
| 4 Week Bleed | 6 Week Bleed |

| | Mean titre | Std. Dev. | Mean titre | Std. Dev. |
|---|---|---|---|---|
| Group | (log₂titre/100) | (log₂titre/100) | (log₂titre/100) | (log₂titre/100) |
| Alum placebo | 0.5 | 1.2 | 0.0 | 0.0 |
| Iscomatrix ™ placebo | 1.0 | 0.0 | 0.0 | 0.0 |
| PCPP placebo (200 μg) | 0.0 | 0.0 | 0.0 | 0.0 |
| DC-Chol placebo (200 μg) | 0.0 | 0.0 | 0.0 | 0.0 |
| RSV Subunit 0.1 μg with no adjuvant | 0.0 | 0.0 | 0.0 | 0.0 |
| RSV Subunit 0.1 μg with Alum | 7.0 | 1.0 | 12.4 | 0.9 |
| RSV Subunit 1 μg with Alum | 8.7 | 0.8 | 11.4 | 0.8 |
| RSV Subunit 10 μg with Alum | 9.7 | 0.8 | 12.3 | 1.0 |
| RSV Subunit 1 μg with Iscomatrix ™ | 8.5 | 0.6 | 13.3 | 0.5 |
| RSV Subunit 10 μg with Iscomatrix ™ | 10.0 | 0.0 | 13.0 | 0.0 |
| RSV Subunit 1 μg with PCPP (200 μg) | 10.2 | 0.8 | 14.0 | 0.7 |
| RSV Subunit 0.5 μg with DC-Chol (200 μg) | 9.7 | 1.4 | 13.0 | 1.0 |

TABLE 7
Lung Virus Titres in Balb/c Mice

| | Mean titre | Std. Dev. |
|---|---|---|
| Group | (log₁₀/g lung) | (log₁₀/g lung) |
| Alum placebo | 4.1 | 0.2 |
| Iscomatrix ™placebo | 3.5 | 0.1 |
| PCPP placebo (200 μg) | 5.2 | 0.2 |
| DC-Chol placebo (200 μg) | 5.0 | 0.3 |
| RSV Subunit 0.1 μg with no adjuvant | 5.3 | 0.1 |
| RSV Subunit 0.1 μg with Alum | <1.7¹ | 1.7 |
| RSV Subunit 1 μg with Alum | <1.7 | 1.7 |
| RSV Subunit 10 μg with Alum | <1.7 | 1.7 |
| RSV Subunit 1 μg with Iscomatrix ™ | <1.7 | 1.7 |
| RSV Subunit 10 μg with Iscomatrix ™ | <1.7 | 1.7 |
| RSV Subunit 1 μg with PCPP (200 μg) | <1.7 | 1.7 |
| RSV Subunit 0.5 μg with DC-Chol (200 μg) | <1.7 | 1.7 |

¹minimal detectable virus titre in assay

TABLE 8
Serum Neutralization Titres in African Green Monkeys

| | 3 Week Bleed | | 5 Week Bleed | | 7 Week Bleed | |
|---|---|---|---|---|---|---|
| | Mean | Std | Mean | Std | Mean | Std. |
| | titre | Dev. | titre | Dev. | titre | Dev. |
| Group | (log₂) | (log₂) | (log₂) | (log₂) | (log₂) | (log₂) |
| Alum placebo | 3.3 | 0.0 | 3.3 | 0.0 | 3.3 | 0.0 |
| Iscomatrix^{™ placebo} | 3.3 | 0.0 | 3.3 | 0.0 | 3.3 | 0.0 |
| RSV Subunit 100 μg | 11.3 | 1.3 | 14.6 | 1.3 | 11.5 | 1.4 |
| with Alum | | | | | | |
| RSV Subunit 100 μg | 10.8 | 0.7 | 15.1 | 0.1 | 11.9 | 0.5 |
| with Iscomatrix ™ | | | | | | |

TABLE 9
Serum Anti-F Titres in African Green Monkeys

| | 3 Week Bleed | | 5 Week Bleed | | 7 Week Bleed | |
|---|---|---|---|---|---|---|
| | Mean | Std | Mean | Std | Mean | Std. |
| | titre | Dev. | titre | Dev. | titre | Dev. |
| | (log₂ | (log₂ | (log₂ | (log₂ | (log₂ | (log₂ |
| | titre/ | titre/ | titre/ | titre/ | titre/ | titre/ |
| Group | 100) | 100) | 100) | 100) | 100) | 100) |
| Alum placebo | 0.0 | 0.0 | 0.0 | 0.0 | 0.0 | 0.0 |
| Iscomatrix ™ placebo | 0.0 | 0.0 | 0.0 | 0.0 | 0.0 | 0.0 |
| RSV Subunit 100 μg | 6.5 | 1.9 | 9.3 | 1.0 | 9.0 | 1.2 |
| with Alum | | | | | | |
| RSV Subunit 100 μg | 5.5 | 1.0 | 9.8 | 0.5 | 9.5 | 1.0 |
| with Iscomatrix ™ | | | | | | |

TABLE 10
Serum Antibodies Directed against RSV A and RSV B GMT and 95% CI

| | | 100 μg dose/adjuvant 1.5 mg | | | Control | | |
|---|---|---|---|---|---|---|---|
| Day | Antibody | GMT | Lower | Upper | GMT | Lower | Upper |
| Day 0 | NA to RSV A | 1987.1 | 1633.5 | 2417.2 | 1818.2 | 1551.5 | 2130.7 |
| Day 0 | NA to RSV B | 1510.4 | 1246.6 | 1830.1 | 1564.1 | 1348 | 1814.8 |
| Day 0 | Anti-F | 72093.5 | 60307 | 86183.6 | 73234.6 | 62631.9 | 85632.2 |
| Day 0 | Anti-G | 69710.9 | 57795.3 | 84083.1 | 76336.9 | 64091.2 | 90922.5 |
| Day 32 | NA to RSV A | 7627.4 | 6298.9 | 9236 | 1731.4 | 1485.7 | 2017.8 |
| Day 32 | NA to RSV B | 4994.6 | 4136.9 | 6030.2 | 1552 | 1331.2 | 1809.3 |
| Day 32 | Anti-F | 311418.1 | 262682.4 | 369195.9 | 73542.3 | 62794 | 86130.3 |
| Day 32 | Anti-G | 193516.7 | 161887.9 | 231325 | 74111 | 62145.5 | 88380.4 |
| Day 60 | NA to RSV A | 7495.5 | 6277.4 | 8950 | 1808 | 1539.1 | 2123.8 |
| Day 60 | Anti-F | 314135.9 | 267418 | 369015.4 | 75367.1 | 64209 | 88464.2 |
| Day 60 | Anti-G | 175019 | 147707.2 | 207380.9 | 80217.7 | 67060 | 959457.1 |
| Day 180 | NA to RSV A | 4718.7 | 3936.5 | 5656.3 | 2276 | 1881.9 | 2752.8 |
| Day 180 | Anti-F | 205150.6 | 174134.9 | 241690.6 | 79623.8 | 66378.5 | 95512.1 |
| Day 180 | Anti-G | 126833.4 | 106591.3 | 150919.5 | 74767.5 | 61397.6 | 91048.9 |

TABLE 11
Greater than or Equal to Two Fold increase antibody titre

| | | 100 μg/adjuvant | | Control | |
|---|---|---|---|---|---|
| Day | Antibody | N | % | N | % |
| Day 32/Day 0 | NA to RSV A | 86 | 76.11 | 1 | 0.93 |
| Day 32/Day 0 | NA to RSV B | 77 | 68.14 | 0 | 0 |
| Day 32/Day 0 | NA to RSV A | 70 | 61.95 | 0 | 0 |
| | and RSV B | | | | |
| Day 32/Day 0 | Anti-F | 92 | 81.42 | 2 | 1.87 |
| Day 32/Day 0 | Anti-G | 70 | 61.95 | 5 | 4.67 |
| Day 60/Day 0 | NA to RSV A | 88 | 80 | 4 | 3.85 |
| Day 60/Day 0 | Anti-F | 97 | 88.18 | 2 | 1.92 |
| Day 60/Day 0 | Anti-G | 62 | 56.36 | 5 | 4.81 |
| Day 180/Day 0 | NA to RSV A | 63 | 60 | 14 | 14 |
| Day 180/Day 0 | Anti-F | 71 | 67.62 | 8 | 8 |
| Day 180/Day 0 | Anti-G | 38 | 36.19 | 7 | 7 |

TABLE 12
Greater than or Equal to Four Fold increase in antibody titre

| | | 100 μg/adjuvant | | Control | |
|---|---|---|---|---|---|
| Day | Antibody | N | % | N | % |
| Day 32/Day 0 | NA to RSV A | 50 | 44.25 | 0 | 0 |
| Day 32/Day 0 | NA to RSV B | 40 | 35.4 | 0 | 0 |
| Day 32/Day 0 | NA to RSV A | 35 | 30.97 | 0 | 0 |
| | and RSV B | | | | |
| Day 32/Day 0 | Anti-F | 52 | 46.02 | 1 | 0.93 |
| Day 32/Day 0 | Anti-G | 32 | 28.32 | 0 | 0 |
| Day 60/Day 0 | NA to RSV A | 49 | 44.55 | 1 | 0.96 |
| Day 60/Day 0 | Anti-F | 52 | 47.27 | 2 | 1.92 |
| Day 60/Day 0 | Anti-G | 28 | 25.45 | 0 | 0 |
| Day 180/Day 0 | NA to RSV A | 24 | 22.86 | 3 | 3 |
| Day 180/Day 0 | Anti-F | 32 | 30.48 | 4 | 4 |
| Day 180/Day 0 | Anti-G | 14 | 13.33 | 3 | 3 |

### REFERENCES

1. Glezen, W. P., Paredes, A. Allison, J. E., Taber, L. H. and Frank, A. L. (1981). J. Pediatr. 98, 708-715.

2. Chanock, R. M., Parrot, R. H., Connors, M., Collins, P. L. and Murphy, B. R. (1992) Pediatrics 90, 137-142.

3. Martin, A. J. Gardiner, P. S. and McQuillin, 3. (1978). Lancel ii, 1035-1038.

4. Robbins, A., and Freeman, P. (1988) Sci, Am. 259, 126-133.

5. Glezen, W. P., Taber, L. H., Frank A. L. and Kasel, J. A. (1986) Am. J. Dis. Child. 140, 143-146.

6. Katz, S. L. Now vaccine development establishing priorities. Vol. 1. Washington: National Academic Press. (1985) pp.397-409.

7. Wertz, G. W., Sullender, W. M. (1992) Biotech. 20, 151-176.

8. McIntosh, K. and Chanopk, R. M., (1990) in Fields Virology (Fields, B. M., and Knipe, D. M. eds.) pp. 1045-1075, Raven Press, Ltd., New York

8A. Dowell, S. F. et al, 1996, J. Infect. Dis. 174:456-462.

9. Collins, P., McIntosh, K., and Chanock, R. M. in “Fields Virology” ed. by Fields, B. N., Knipe, D. M., and Howley, P. M., Lippincott-Raven Press, New York, (1996) pp. 1313-1351.

10. Walsh, E. E., Hall, C. B., Briselli, M., Brandiss, M. W. and Schleiger, J. J. (1987) J. Infect Dis. 155,1198-1204.

11. Walsh, E. E., Hruska, J. (1983) J. Virol. 47, 171-177.

12. Levine, S., Kleiber-France, R., and Paradiso, P. R. (1987) J. Gen. Virol. 69, 2521-2524.

13. Anderson, L. J. Hierholzer, J. C., Tsou, C., Hendry, R. M., Fernie, B. F., Stone, Y. and McIntosh, K. (1985), J. Infect. Dis. 151, 626-633.

14. Jobnson, P. R., Olmsted, R. A., Prince, G. A., Murphy, B. R., Alling, D. W., Walsh, E. E. and Collins, P. L. (1987) J. Virol. 61 (10), 3163-3166.

15. Cherrie, A. H., Anderson, K., Wertz, G. W., and Openshaw, P. J. M. (1992) J. Virology 66, 2102-2110.

16. Kim, H. W., Canchola, J. G., Brandt, C. D., Pyles, G., Chanock, R. M. Jensen, K., and Parrott, R. H. (1969) Amer. J., Epidemiology 89, 422-434.

17. Firedewald, W. T., Forsythe B. R, Smith, C. B., Gharpure, M. S., and Chanock, R. M. (1968) JAMA 204, 690-694.

18. Walsh, E. E., Brandriss, M. W., Schlesinger, J. J. (1985) J. Gen. Virol, 66, 409-415.

19. Walsh, E. E., Schlesinger, J. J. and Brandriss, M. W. (1984) J. Gen. Virol. 65, 761-766.

20. Routledge, E. G., Willcocks, M. M., Samson, A. C. R, Morgan, L., Scott, R., Anderson, J. J., and Toms, G. L. (1988) J. Gen. Virology 69, 293-303.

21. Fulginiti, V. A., Eller, J. J., Sieber, O. F., Joyner, I. W., Minamitani, M. and Meiklejohn, G. (1969) Am J. Epidemiol. 89 (4), 435-448.

22. Chin, J., Magoffn, R L., Shearer, L. A., Schieble, J. H. and Lennette, E. H. (1969) Am. J. Epidemiol. 89 (4), 449-463.

23. Kapikian, A. Z., Mitchell, R. H., Chanock R. M., Shvedoff, R A. and Stewart, C. E. (1969) Am. J. Epidemiol. 89 (4), 405-421.

24. Kim, H. W., Arrobio, J. O., Pyles, G., Brandt, C. D. Camargo, E., Chanock, R. M. and Parrott, R. H. (1971) Pediatrics 48, 745-755.

25. Wright, P. F., Belshe, R. B., Kim, H. W., Van Voris, L. P. and Chanock, r. M. (1982) Infect. Immun. 37 (1), 397-400.

26. Wright P. F., Chinozaki, T. and Fleet, W. (1976) J, Pediatr. 88, 931-936.

27. Belshe, R B., Van Voris, P. and Mufson, M. A. (1982) J. Infect. Dis. 145, 311-319.

28. Murphy, B. R., Prince, G. A., Walsh, E. B., Kim, H. W., Parrott, R. H., Hemming V. G., Rodriguez, W. J., and Chanock, R. M., J. Clin. Microbiol. (1986), 24(2), 197-202.

29. Connors, M., Collins, P. L., Firestone, C. Y., Sotnikov, A V., Waitze, A., Davis, A. R., Hung, P. P., Chanock, R. M., Murphy, b. (1992) Vaccine, 10, 475-484.

30. Prince, G. A., Jenson, A. B., Hemming, V. G., Murphy, E. R., Walsh, E. E., Horswood, R. L. and Chanock, R. L. (1986b) J. Virol. 57 (3), 721-728.

31. Piedra, P. A., Camussi, F. and Ogra, P. L. (1989) J. Gen. Virol. 70, 325-333.

32. Walsh, E. E., Hall, C. B., Briselli, M., Brandiss, M. W. and Schlesinger, J. J. (1987) J. lect. Dis. 155 (6), 1198-1204.

33. Prince et al, Am. J. Pathol. 93:771-791.

What we claim is:

1. A mixture of purified fusion (F) protein, attachment (G) protein and matrix (M) protein of respiratory syncytial virus (RSV).

2. The mixture of claim 1 wherein said fusion (F) protein comprises multimeric fusion (F) proteins.

3. The mixture of claim 2 wherein, when analyzed under non-reducing conditions, said multimeric fusion (F) protein includes heterodimers of molecular weight approximately 70 kDa and dimeric and trimeric forms.

4. The mixture of claim 1 wherein, when analyzed under non-reducing conditions, said attachment (G) protein comprises G protein of molecular weight approximately 95 kDa and G protein of molecular weight approximately 55 kDa and oligomeric G protein.

5. The mixture of claim 1 wherein, when analyzed by SDS-PAGE under non-reducing conditions, said matrix (M) protein comprises M protein of molecular weight approximately 28 to 34 kDa.

6. The mixture of claim 1 wherein, when analyzed by reduced SDS-PAGE analysis, said fusion (E) protein comprises F₁ of molecular weight approximately 48 kDa and F₂ of molecular weight approximately 23 kDa, said attachment (G) protein comprises a G protein of molecular weight approximately 95 kDa and a G protein of molecular weight approximately 55 kDa, and said matrix (M) protein comprises an M protein of approximately 31 kDa.

7. The mixture of claim 1 wherein said F, G and M proteins are present in the relative proportions of:
F from about 35 to about 70 wt %
G from about 2 to about 30 wt %
M from about 10 to about 50 wt %.

8. The mixture of claim 7, wherein, when analyzed by SDS-PACE under reducing conditions and silver stained, the ratio of F₁ of molecular weight approximately 48 kDa to F₂ of molecular weight approximately 23 kDa is between 1:1 to about 2:1 by scanning densitometry.

9. The mixture of claim 7 which is at least about 75% pure.

10. The mixture of claim 1 which is devoid of monoclonal antibodies.

11. The mixture of claim 1 which is devoid of lentil lectin and concanavalin A.

12. The mixture of claim 1 wherein said RSV proteins are non-denatured.

13. The mixture of claim 1 wherein said RSV proteins are from one or both of subtypes RSV A and RSV B.

14. A coisolated and copurified mixture of non-denatured proteins of respiratory syncytial virus (RSV), consisting essentially of the fusion (F) protein, attachment (G) protein and matrix (N) protein of RSV, wherein the mixture is free from lectins and is free from monoclonal antibodies.

15. An Immunogenic composition comprising an immunoeffective amount of the mixture of claim 1.

16. The immunogenic composition of claim 15 formulated as a vaccine for in vivo administration to a host to confer protection against RSV.

17. The immunogenic composition of claim 15 further comprising at least one adjuvant or at least one immunomodulator.

18. The immunogenic composition of claim 17 wherein the at least one adjuvant is selected from the group consisting of aluminum phosphate, aluminum hydroxide, QS21, Quil A or derivatives or components thereof, calcium phosphate, calcium hydroxide, zinc hydroxide, a glycolipid analog, an octodecyl ester of an amino acid, a muramyl dipeptide, a lipoprotein, polyphosphazene, ISCOM matrix, DC-chol, DDA and bacterial toxins or derivatives thereof.

19. The immunogenic composition of claim 16 wherein the host is a primate.

20. The immunogenic composition of claim 19 wherein the primate is a human.

21. The immunogenic composition of claim 15 further comprising at least one additional immunogen.

22. The immunogenic composition of claim 21 wherein said at least one additional immunogen comprises at least one human parainfluenza virus (PIV) protein selected from the group consisting of PIV-1, PIV-2 and PIV-3.

23. A method of generating an immune response in a host, comprising administering thereto an immunoeffective amount of the immunogenic composition of claim 15.

24. The method of claim 23 wherein said immunogenic composition is formulated as a vaccine for in vivo administration to the host and said administration to the host confers protection against respiratory syncytial virus.

25. A method for producing a vaccine for protection against respiratory syncytial virus (RSV), comprising:
administering the immunogenic composition of claim 15 to a test host to determine the amount of and frequency of administration thereof to confer protection against disease caused by RSV; and
formulating the immunogenic composition in a form suitable for administration to a treated host in accordance with said determined amount and frequency of administration.

26. The method of claim 25 wherein the treated host is a human.

27. A method of producing monoclonal antibodies specific for fusion (F) protein, attachment (G) protein and matrix (M) protein of respiratory syncytial virus (RSV), comprising:
(a) administering an immunogenic composition of claim 15 to at least one mouse to produce at least one immunized mouse;
(b) removing B-lymphocytes from the at least one immunized mouse;
(c) fusing the B-lymphocytes from the at least one immunized mouse with myeloma cells, thereby producing hybridomas;
(d) cloning the hybridomas which produce a selected anti-RSV protein antibody;
(e) culturing the selected anti-RSV protein antibody-producing clones; and
(f) isolating anti-RSV protein antibodies from the selected cultures.

28. A method of producing a coisolated and copurified mixture of proteins of respiratory syncytial virus (RSV), which comprises:
growing RSV on cells in a culture medium;
separating the grown virus from the culture medium;
solubilizing at least the fusion (F) protein, attachment (G) protein and the matrix (M) protein from the separated virus; and
coisolating and copurifying the solubilized RSV proteins.

29. The method of claim 28 wherein said coisolation and copurification are effected by:
loading the solubilized proteins onto an ion-exchange matrix; and
selectively coeluting the F, G and M proteins from the ion-exchange matrix.

30. The method of claim 29 wherein said ion-exchange matrix is a hydroxyapatite matrix.

31. The method of claim 28 wherein said grown virus is washed with urea to remove contaminants without substantial removal of F, G and M proteins prior to solubilization step.

32. The method of claim 29 including contacting said eluted F, G and M proteins with an anion exchange matrix to remove any residual DNA.

33. A method of determining the presence in a sample of antibodies specifically reactive with a fusion (F) protein, attachment (G) protein or matrix (M) protein of respiratory syncytial virus (RSV), comprising the steps of:
(a) contacting the sample with the mixture of claim 1 to produce complexes comprising a respiratory syncytial virus protein and any said antibodies present in the sample specifically reactive therewith; and
(b) determining production of the complexes.

34. A method of determining the presence in a sample of an F, G or M protein of respiratory syncytial virus, comprising the steps of:
(a) immunizing a subject with the immunogenic composition of claim 15 to produce antibodies specific for F, G and M proteins of RSV;
(b) contacting the sample with the antibodies to produce complexes comprising any RSV protein present in the sample and said protein specific antibodies; and
(c) determining production of complexes.

35. A diagnostic kit for determining the presence of antibodies in a sample specifically reactive with a fusion (F) protein, attachment (G) protein or a matrix (M) protein of respiratory syncytial virus comprising:
(a) a mixture of claim 1;
(b) means for contacting the immunogenic composition with the sample to produce complexes comprising a respiratory syncytial virus protein and any said antibodies present in the sample; and
(c) means for determining production of the complexes.

36. A mixture of purified fusion (F) protein, attachment (G) protein and matrix (M) protein of respiratory syncytial virus (RSV) for use as a pharmaceutical substance in a vaccine against disease caused by infection with respiratory syncytial virus.
